(19) United States
(12) Patent Application Publication
(10) Pub. No.: US 2020/0197603 A1
(43) Pub. Date: Jun. 25, 2020

(54) MEDICAMENT DELIVERY DEVICE

(71) Applicant: Owen Mumford Limited, Woodstock, Oxfordshire (GB)

(72) Inventors: Toby Cowe, Woodstock, Oxfordshire (GB); Tara Preston, Woodstock, Oxfordshire (GB)

(21) Appl. No.: 16/634,668

(22) PCT Filed: Jul. 26, 2018
(86) PCT No.: PCT/GB2018/052107

§ 371 Date: Jan. 28, 2020

(30) Foreign Application Priority Data
Jul. 28, 2017 (GB) 1712184.9

(51) Int. Cl. A61M 5/142; A61M 5/145; A61M 5/148

(52) U.S. Cl. CPC A61M 5/14248; A61M 5/14593; A61M 2209/088; A61M 2005/14252; A61M 2005/14268; A61M 5/148

(57) ABSTRACT

A medicament delivery device for delivering a dose of medicament into an injection site comprises an expandable reservoir (130) for receiving the medicament, a holder (102) for receiving the reservoir (130), attachment means (104, 110) for securing the holder (102) to the injection site, a cannula (174) moveable from a stowed position in which the cannula (174) does not project from the device (100) to a deployed position in which the cannula (174) provides fluid connection between the reservoir (130) and the injection site, and an elastic element (106). In use, the elastic element (106) is held under strain by the medicament in the reservoir (130) and delivery of the medicament through the cannula (174) is driven by relaxation of the elastic element (106) when the cannula (174) is in the deployed position. The device is preferably in the form of an arm band or a patch.

### FIELD OF THE INVENTION

The present invention relates to medicament delivery devices. In particular, but not exclusively, the invention relates to wearable devices for the delivery of relatively large volumes of medicaments by injection.

### BACKGROUND TO THE INVENTION

A wide range of devices have been developed for the delivery of medicaments by subcutaneous infusion or injection. One area of particular interest is devices that are suitable for use outside a clinical environment by a patient or a non-medically trained individual.

Some existing devices, commonly known as auto-injectors, are designed for the automated injection of a dose of medicament. Such devices typically include a deployable needle, a medicament container in the form of a syringe barrel, a drive spring for driving a stopper of the container, and a trigger arrangement that, when operated, causes the needle to emerge from the device to enter the injection site and releases the drive spring to drive the stopper along the syringe barrel to expel the medicament into the injection site. Such devices are relatively simple and low-cost, and are typically intended for disposal after use.

Auto-injector devices typically have a linear pen-type configuration and are arranged so that, to perform an injection, one end of the device is held against the injection site and then the trigger arrangement is activated. The device must be held in position against the injection site until delivery of the medicament is complete.

The time required to deliver a dose of medicament increases with the volume to be delivered and the viscosity of the medicament, and decreases with the size of the needle or cannula, and the force applied to expel the medicament. For patient comfort, it is generally desirable to minimise the size of the cannula and the expulsion force. A relatively low expulsion force is also preferred for device reliability. Accordingly, auto-injector devices are best suited to applications in which the required dose of medicament can be delivered over a relatively short time, typically a few seconds. When a large quantity of medicament and/or a high viscosity medicament is to be delivered, the injection time required often exceeds that for which an auto-injector device can be comfortably and reliably held against an injection site. As a result, auto-injectors are generally unsuitable for the delivery of dose volumes in excess of around 1-2 mL and for use with medicaments of relatively high viscosity.

Devices for the delivery of larger volumes of medicaments (typically 2 mL and above) over longer periods of time (typically 1 minute or longer) have also been developed. Typically, in such devices, the stopper of a medicament cartridge is displaced by a motor-driven plunger at a pre-programmed rate. Some such devices are designed to be attached to the injection site by an adhesive patch whilst medicament delivery takes place. These devices are relatively complex and expensive, and are not therefore generally intended as single use, disposable devices. Furthermore, due to their technical complexity, such devices can be relatively heavy and bulky, making them uncomfortable if attached a patient over an extended period of time.

It would therefore be desirable to provide devices suitable for delivering a relatively large volume of medicament over a relatively long period of time that are simple, reliable, inexpensive and optimised for a single use.

### SUMMARY OF THE INVENTION

Against that background, and from a first aspect of the present invention, a medicament delivery device for delivering a dose of medicament into an injection site is provided, the device including an expandable reservoir for receiving the medicament, a holder for receiving the reservoir, attachment means for securing the holder to the injection site, a cannula moveable from a stowed position in which the cannula does not project from the device to a deployed position in which the cannula provides fluid connection between the reservoir and the injection site, and an elastic element wherein, in use, the elastic element is held under strain by the medicament in the reservoir, and delivery of the medicament through the cannula is driven by relaxation of the elastic element when the cannula is in the deployed position.

Devices according to the invention can be comfortably worn by a patient over an extended period to allow the delivery of larger doses and/or the use of higher viscosity medicaments compared with prior art devices. In particular, the use of an elastic element in combination with an expandable reservoir provides a simple, lightweight means for driving delivery of a relatively large volume of medicament, and avoids the need for drive springs or motors.

With this arrangement, delivery of the medicament can be driven by the elastic strain energy that is generated by the medicament volume in the reservoir acting on the elastic element to cause an elastic strain in the elastic element. Said another way, the elastic element is arranged to pressurise the medicament in the reservoir by compressing the reservoir.

Preferably, the reservoir and the elastic element are separate components. In this case the elastic element does not form part of the reservoir itself (so for example the elastic element does not form a wall of the reservoir), but rather is a physically separate component that cooperates with the reservoir to drive delivery of the medicament from the reservoir. The reservoir and the elastic element may be of different materials. The reservoir may comprise a wall that at least partially defines an interior of the reservoir for receiving the medicament, and the elastic element may be disposed adjacent to the wall.

The reservoir may for example have a capacity of between 2 mL and 50 mL. Preferably, the reservoir has a capacity of between 3 mL and 10 mL. The reservoir may comprise a flexible bag.

The elastic element is preferably of an elastomeric material. For example, the elastic element may comprise a thermoplastic elastomer material. The holder may comprise the elastic element.

The elastic element may be stretchable, such that the elastic element is held under tensile strain by the medicament in the reservoir. Alternatively, the elastic element may be compressible, such that the elastic element is held under compressive strain by the medicament in the reservoir.

Preferably, the cannula comprises a flexible needle. The cannula may be of a superelastic material, such as nitinol.

The cannula may comprise an orifice positioned such that the orifice is disposed within the reservoir when the cannula is in the deployed position. In this way, a fluid connection between the reservoir and the cannula can be opened automatically upon movement of the cannula to the deployed position, without the need for a valve or other relatively complex connection. The cannula may comprise a tip, and the orifice may be remote from the tip, in a shaft part of the cannula.

Preferably, when in the stowed position, the cannula does not encroach into or otherwise cooperate with the reservoir. The cannula may be arranged to pierce the reservoir upon movement of the cannula from the stowed position to the deployed position. In this way, the sterility of the interior of the reservoir can be maintained, without the need for a valve or port, until the device is used.

The device may comprise a cannula insertion mechanism operable to move the cannula from the stowed position to the deployed position. Preferably, the cannula insertion mechanism is further operable to retract the cannula from the deployed position. Conveniently, the reservoir may extend between the cannula insertion mechanism and a contact surface of the device.

The cannula insertion mechanism may comprise a power spring and a crank arrangement driven by the power spring and coupled to the cannula for moving the cannula between the stowed and deployed positions. In this way, a particularly compact arrangement can be provided. The device may comprise a trigger component having a first position in which the trigger component blocks movement of the crank arrangement with the cannula in the stowed position, and a second position in which the trigger component blocks movement of the crank arrangement with the cannula in the deployed position.

The attachment means may comprise an adhesive layer for securing the device to the injection site. Alternatively, or in addition, the attachment means may comprise an adjustable strap.

In some embodiments, the device is elongate and flexible to be worn around a limb. In this case, the elastic element may comprise a tubular band, and at least a part of the reservoir may extend within the band.

In other embodiments, the device comprises a wearable patch. The elastic element may comprise an elastic sheet. The holder may comprise a chassis, and at least a part of the reservoir may extend between the chassis and the elastic sheet.

The device may be arranged so that the amount of elastic strain energy stored in the elastic element varies with position, so that the force applied to the reservoir also varies with position. Thus the elastic element may be arranged to apply a driving force to the reservoir that varies with distance from the cannula. For example, the elastic element may have a variable thickness. The thickness of the elastic element may increase with distance from the cannula when the cannula is in the deployed position, so that the reservoir empties first from the parts of the reservoir furthest from the cannula to ensure complete delivery of the medicament dose. In another arrangement, the medicament in the reservoir applies a non-uniform strain to the elastic element. For example, the strain required to accommodate the reservoir when filled with medicament may increase with distance from the cannula.

The device may comprise a filling port for admitting medicament to the reservoir. For example, the filling port may comprise a one-way valve. The filling port may be arranged for cooperation with a filling syringe, and may for example incorporate a slip-tip (Luer slip) or Luer lock connector.

To this end, the invention also extends, in a second aspect, to a kit comprising a device according to the first aspect of the invention and a filling syringe for transferring medicament to the reservoir of the device.

In a further aspect, the invention comprises a method for delivering a dose of medicament to an injection site, comprising transferring the medicament to a reservoir of a device to cause elastic deformation of an elastic element, securing the device to the injection site, and deploying a cannula from the device to form a fluid connection between the reservoir and the injection site such that relaxation of the elastic element drives delivery of the medicament through the cannula.

Preferred and/or optional features of each aspect of the invention may also be used, alone or in suitable combination, in the other aspects of the invention also.

### BRIEF DESCRIPTION OF THE DRAWINGS

Embodiments of the present invention will now be described, by way of example only, with reference to the accompanying drawings, in which like reference numerals are used for like parts, and in which:

FIG. 1 is a perspective view of a medicament delivery device according to a first embodiment of the present invention in a worn and filled configuration;

FIG. 2 is a perspective view of the device of FIG. 1 in an open empty configuration;

FIG. 3 is a side view of the device of FIG. 1 in the open empty configuration;

FIG. 4 is a cross-sectional side view of the device of FIG. 1 in the open empty configuration;

FIG. 5 is an exploded perspective view showing holder and reservoir components of the device of FIG. 1;

FIG. 6 is an exploded perspective view showing filling end parts of the holder and reservoir components of FIG. 5;

FIG. 7 is a sectioned perspective view of a filling port of the device of FIG. 1;

FIG. 8 is a perspective view of the device of FIG. 1 in the worn configuration and with a syringe engaged with the filling port;

FIGS. 9(a) and 9(b) are cross-sectional side views of the device of FIG. 1 in the worn configuration before and after filling, respectively;

FIG. 10 is an exploded perspective view showing components of a cannula insertion mechanism of the device of FIG. 1;

FIG. 11 is a cross-sectional side view of the mechanism of FIG. 10 with the cannula in a stowed position;

FIGS. 12(a) and 12(b) are perspective views of the mechanism of FIG. 10 with the cannula in the stowed position;

FIG. 13 is a cross-sectional side view of the mechanism of FIG. 10 with the cannula in a deployed position;

FIGS. 14(a) and 14(b) are perspective views of the mechanism of FIG. 10 with the cannula in the deployed position;

FIG. 15 is a perspective view of the device of FIG. 1 with the cannula in the deployed position;

FIG. 16 is a cross-sectional view of part of the device of FIG. 1 with the cannula in the deployed position;

FIG. 17 is a perspective view of a medicament delivery device according to a second embodiment of the present invention;

FIG. 18 is another perspective view of the device of FIG. 17;

FIG. 19 is a sectioned perspective view of the device of FIG. 17;

FIG. 20 is an exploded perspective view of the device of FIG. 17;

FIG. 21 is a perspective view of the device of FIG. 17 with a syringe engaged with a filling port; and

FIGS. 22(a) and 22(b) are cross-sectional side views of the device of FIG. 17 after filling and with a cannula in stowed and deployed positions, respectively.

### DESCRIPTION OF THE PREFERRED EMBODIMENTS

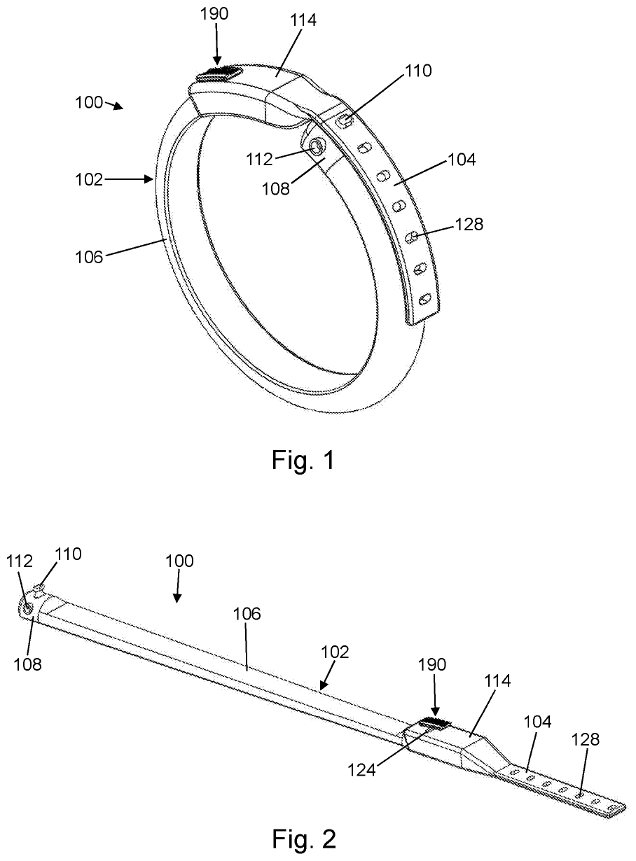

FIGS. 1 to 4 show a medicament delivery device according to a first embodiment of the present invention. The device 100 is in the form of an arm band comprising an elongate body or holder 102 that can be wrapped around a user's arm (or other limb) and secured with an integral strap 104. FIG. 1 shows the device 100 in an applied or worn configuration, where the holder 102 is wrapped around the user's arm in a generally circular configuration and the strap 104 is fastened, and FIGS. 2 to 4 show the device 100 in an open, linear configuration.

The holder 102 comprises a band 106 in the form of a flattened tube that extends between first and second ends. The first end of the band 106 is attached to a fastener end 108 of the holder 102 having an outwardly-projecting catch 110 and a filling port 112. The second, opposite end of the band 106 is attached to a housing 114 for a cannula insertion mechanism 120 (see FIG. 4), and a trigger component 190 of the insertion mechanism 120 extends through an aperture 124 in the outer face of the housing 114. An inside surface of the housing 114 provides a contact surface 126 of the device 100 to be placed against an injection site, in use. The strap 104 is attached to and extends away from the housing 114 so that, when the device 100 is wrapped around a user's arm, one of a plurality of notches or holes 128 in the strap 104 can be fastened to the catch 110 of the fastener end 108 to secure the device 100 in position, with the contact surface 126 against the injection site.

The band 106, and optionally the strap 104, are formed from an elastic, stretchable material, such as a thermoplastic elastomer material, which can be stretched as it is wrapped around the arm to ensure that the device 100 is securely held in place. Conveniently, the fastener end 108 and the housing 114 are made from the same or a similar material, and the parts of the holder 102 may be co-moulded or joined by welding, adhesive or another suitable method.

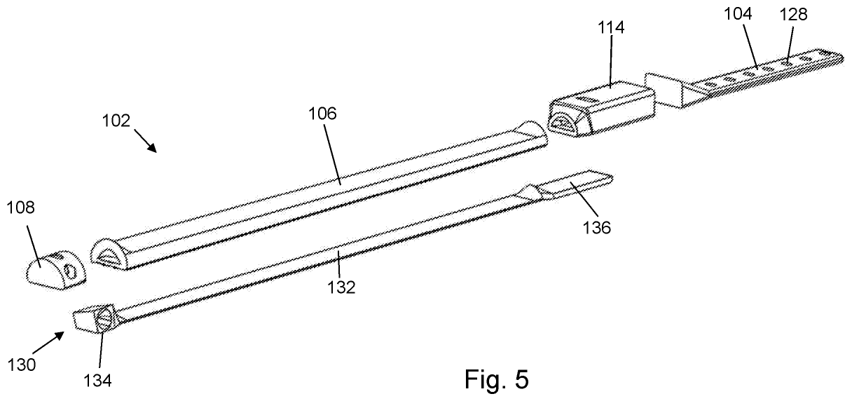

Referring to FIG. 4 and additionally to FIG. 5, the holder 102 accommodates a medicament reservoir 130. The reservoir 130, which is shown separately from the holder 102 in FIG. 5, comprises a flexible bag having an elongate main part 132 in the form of a flattened tube that extends within the band 106 of the holder 102. At a first end of the main part 132, the reservoir bag 130 is moulded to form an enlarged filling end termination 134, which is housed within the fastener end 108 of the holder 102. At a second, opposite end of the main part 132, the reservoir bag 130 is moulded to form an enlarged end chamber 136 that is housed in a slot-like cavity 138 in the housing 114 of the holder 102, beneath the cannula insertion mechanism 120.

The reservoir bag 130 is preferably of a thermoplastic material. Suitable materials include, but are not limited to, ethylene vinyl acetate (EVA), polypropylene (PP) and copolyester elastomers. The reservoir bag 130 forms a wall of the reservoir that encloses an interior or the reservoir for receiving the medicament.

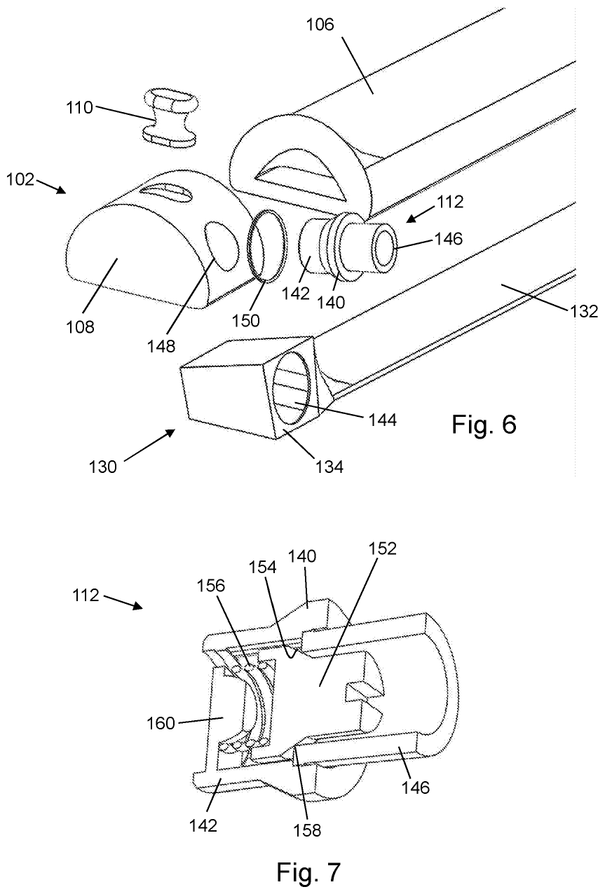

FIG. 6 is an enlarged exploded view showing the fastener end 108 of the holder 102 and the filling end termination 134 of the reservoir bag 130. The filling port 112 comprises a valve 140 that allows medicament to be admitted to the bag 130. The valve 140 may be of a known design. In this example, the valve 140 comprises a two-part, generally tubular valve housing. An inner part 142 of the valve housing extends into an opening 144 in the filling end termination 134 of the reservoir bag 130. An outer, smaller diameter part 146 of the valve housing projects through an aperture 148 in the fastener end 108 of the holder 102. The outer part 146 of the valve housing is dimensioned to accept the tip of a slip-tip (Luer slip) syringe. An O-ring 150 is provided to form a seal between the valve 140 and the reservoir bag 130.

As shown most clearly in FIG. 7, the valve 140 includes a valve element 152 having a frustoconical surface 154 that is biased by a spring 156 to seat against a valve seat 158 formed by an inside end surface of the outer part 146 of the valve housing. The spring 156 acts between the valve element 152 and a spring seat 160 that extends across the inner part 142 of the valve housing.

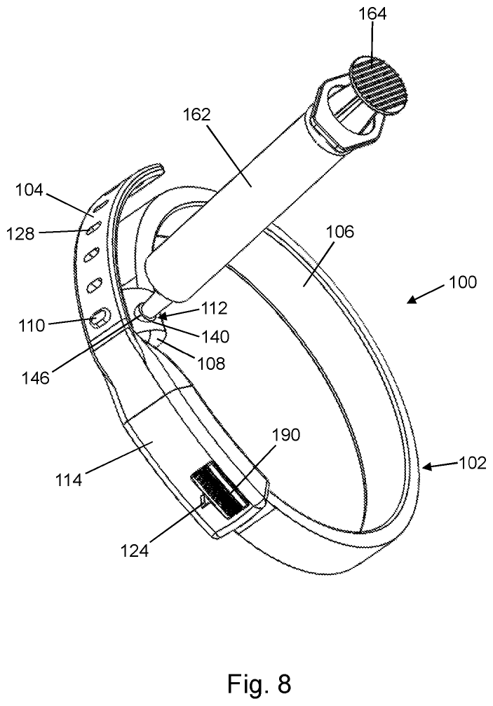

The valve 140 is configured so that the valve element 152 is normally kept seated against the valve seat 158. As shown in FIG. 8, to fill the device 100 with medicament, a slip-tip filling syringe 162 containing the medicament is engaged with the outer part 146 of the valve housing. The plunger 164 of the filling syringe 162 is depressed to transfer medicament from the syringe 162 to the interior of the reservoir bag 130, with the valve element 152 moving away from the valve seat 158 under the pressure of the inflowing medicament. After filling is complete, the valve element 152 moves back into engagement with the valve seat 158 to close the valve 140, and the filling syringe 162 can be removed. The valve 140 therefore preserves the sterility of the interior of the reservoir bag 130 before filling, and prevents leakage of medicament from the device 100 after filling.

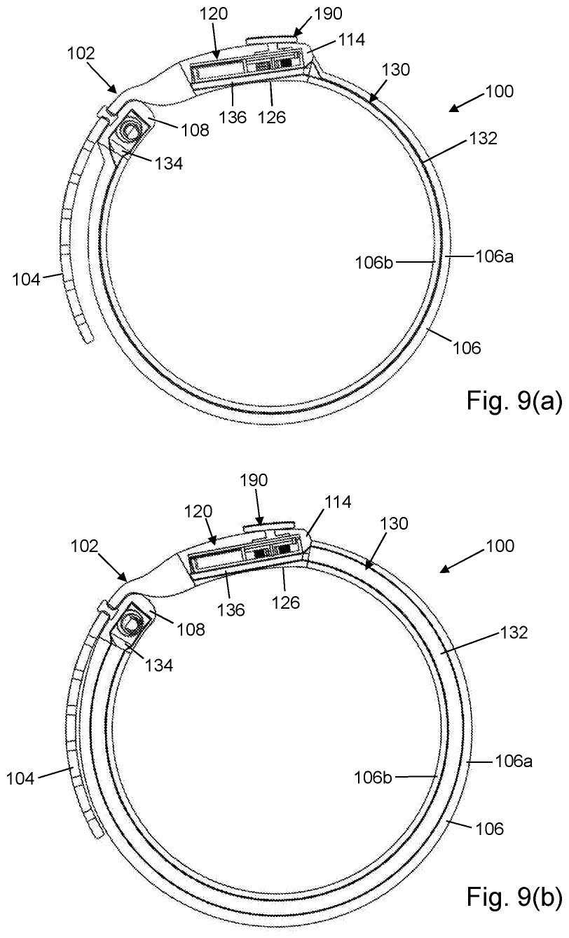

Referring to FIG. 9, transfer of medicament to the device 100 causes the reservoir bag 130 to expand from an initial state as shown in FIG. 9(a) to an expanded state as shown in FIG. 9(b). In the initial state, the main part 132 of the bag 130 is held substantially flat between an outer wall 106a and an inner wall 106b of the band 106 of the holder 102. In the expanded state the main part 132 of the bag 130 adopts a tubular configuration, with the outer and inner walls 106a, 106b of the band 106 moving apart to accommodate the medicament. As can be seen in FIG. 9, the outer wall 106a of the band 106 has a decreasing thickness moving from the fastener end 108 of the holder 102 towards the housing 114. In the illustrated example, the inner wall 106b of the band 106 has a substantially constant thickness. Optionally, the inner wall 106b may be reinforced, for example with an inelastic strip of a suitable material, such as spring steel, or may be substantially thicker than the outer wall 106a, so that deformation of the inner wall 106b is constrained or reduced and the volume of the medicament is substantially accommodated by stretching of the outer wall 106a. In other arrangements, the inner wall 106b may have a varying thickness, in addition to or instead of the outer wall 106a.

As the reservoir bag 130 expands during filling, the material of the band 106 of the holder 102 stretches elastically to accommodate the increasing volume of the reservoir bag 130. Thus the medicament in the bag 130 is pressurised by the compressive force acting on the bag 130 as a result of elastic stretching of the band 106 of the holder 102. Said another way, the band 106 is held under elastic strain by the medicament in the bag 130.

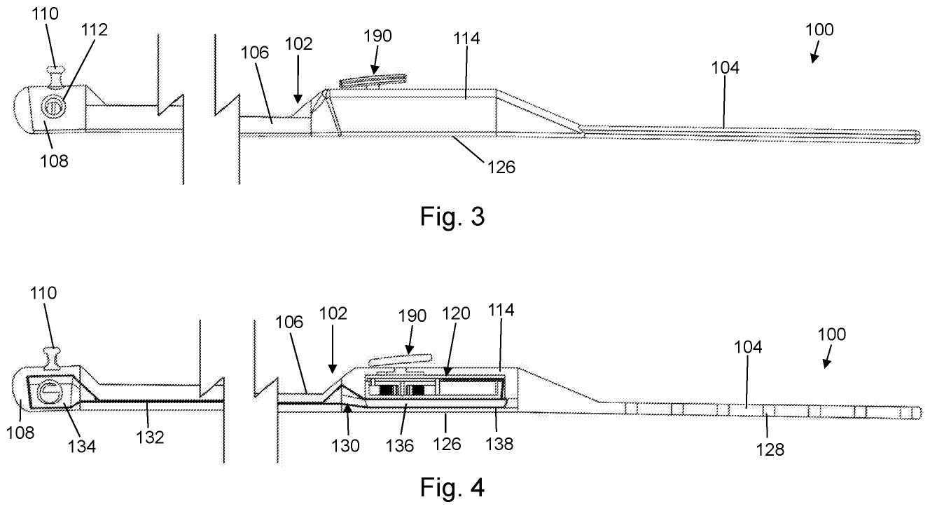

Although FIG. 8 shows the device 100 being filled when in the worn configuration (i.e. when fastened to the patient's arm), it is also possible to fill the device 100 when in the open configuration, before the device 100 is fastened around the arm. In either case, the device 100 is positioned during fastening to the arm so that the contact surface 126 (see FIGS. 3, 4 and 9; not visible in FIG. 8) is held against the desired injection site. Optionally, the contact surface 126 may carry a non-slip feature, or an adhesive layer (not shown) to help secure the contact surface 126 against the injection site.

Once the filled device 100 is in position on the arm, delivery of the medicament to the patient is triggered by operation of the cannula insertion mechanism 120, as will now be described.

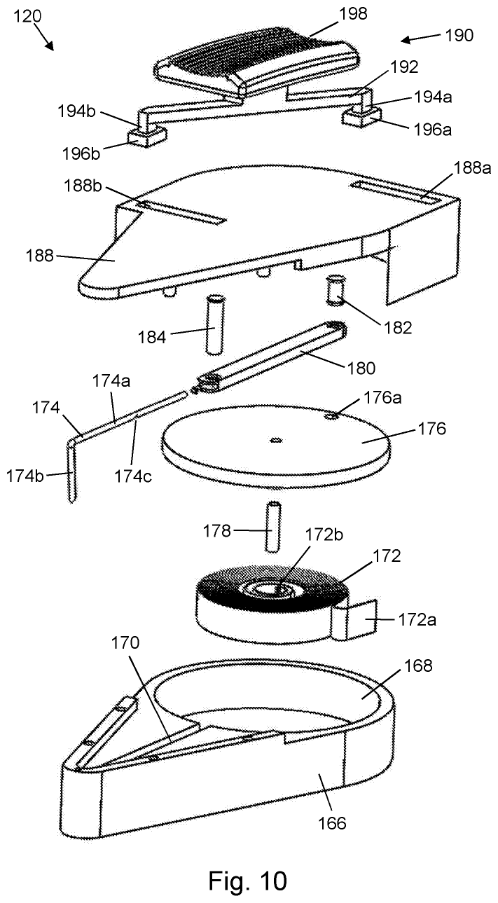

Referring to FIGS. 10, 11, and 12, the cannula insertion mechanism comprises a guide body 166 having a generally cylindrical spring chamber 168 and a guide slot 170 that extends radially outwards from the spring chamber 168 along a tapering part of the guide body 166. A power spring (flat-wound spring) 172 is mounted in the spring chamber 168.

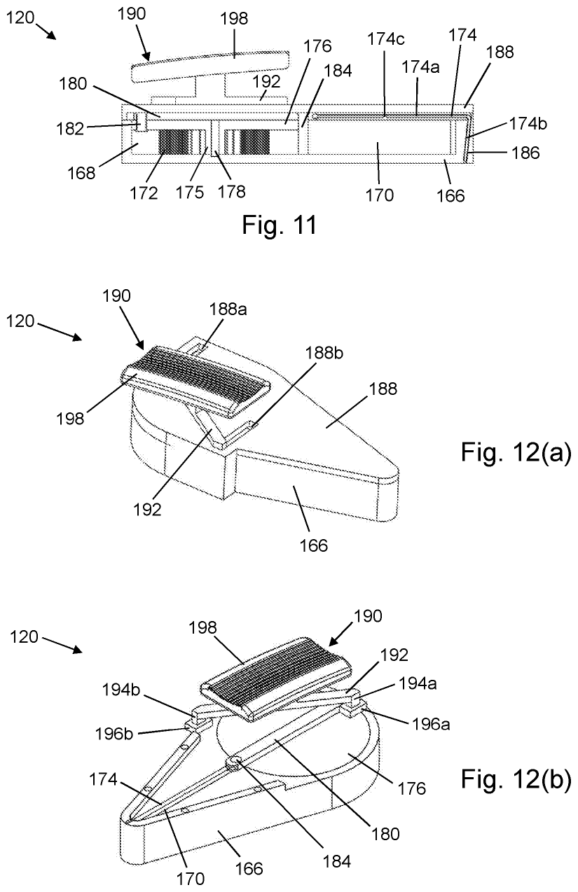

The power spring 172 drives insertion and retraction movement of a cannula 174 by way of a crank arrangement. An outer end termination 172a of the spring 172 is secured to an inside wall of the spring chamber 168. An inner end termination 172b of the spring 172 is engaged with a tubular drive axle 175 (see FIG. 11) that extends downwardly from a disc 176. The disc 176 is rotatably mounted in the spring chamber 168, above the power spring 172, with the axle 175 received on an upstanding spindle 178 attached to the guide body 166. The spring 172 biases the disc 176 for clockwise rotation (in the illustrated orientation) with respect to the guide body 166.

A shaft 180 is disposed on the top surface of the disc 176. A first end of the shaft 180 is connected to the disc 176 by a crank pin 182 that engages with a hole 176a close to the periphery of the disc 176. A second, opposite end of the shaft 180 is engaged with a guide pin 184 that is received in the guide slot 170 of the guide body 166. The cannula 174, which comprises a flexible, tubular needle, is attached to and extends away from the second end of the shaft 180, so that a first part 174a of the cannula 174 extends parallel to and above the guide slot 170. A second part 174b of the cannula 174 extends downwardly through a cannula channel 186 (see FIG. 11) formed in the guide body 166 beyond the end of the guide slot 170. In this example, the channel 186 extends at an inclined angle within the guide body 166 so that the angle formed between the first and second parts 174a, 174b of the cannula 174 is approximately 85 degrees.

The mechanism 120 is closed by a cover 188, which encloses the cannula 174, the shaft 180, the disc 176 and the associated components. A trigger component 190 is disposed on top of the cover 188. As can be seen most clearly in FIG. 10, the trigger component 190 includes a frame 192 that extends diagonally with respect to the guide slot 170. Each end of the frame comprises a leg 194a, 194b that extends through a respective lateral slot 188a, 188b in the cover 188. Each leg 194a, 194b terminates in a respective blocking formation 196a, 196b. The frame 192 also extends upwards, through the slot 124 in the housing 114 (see FIG. 5), and is attached to a slider or button 198. A detent or catch mechanism (not shown) is provided to retain the trigger component 190 in either a first position or a second position, so that movement of the trigger component 190 between the first and second positions requires the user to apply a suitable lateral force to the button 198.

Referring now to FIG. 12(b), in which the cover 188 has been omitted for clarity, in the first position the trigger component 190 is disposed so that a first one 196a of the blocking formations abuts the first end of the shaft 180 to prevent clockwise rotation of the disc 176. In this condition, the cannula 174 remains stowed within the guide body 166.

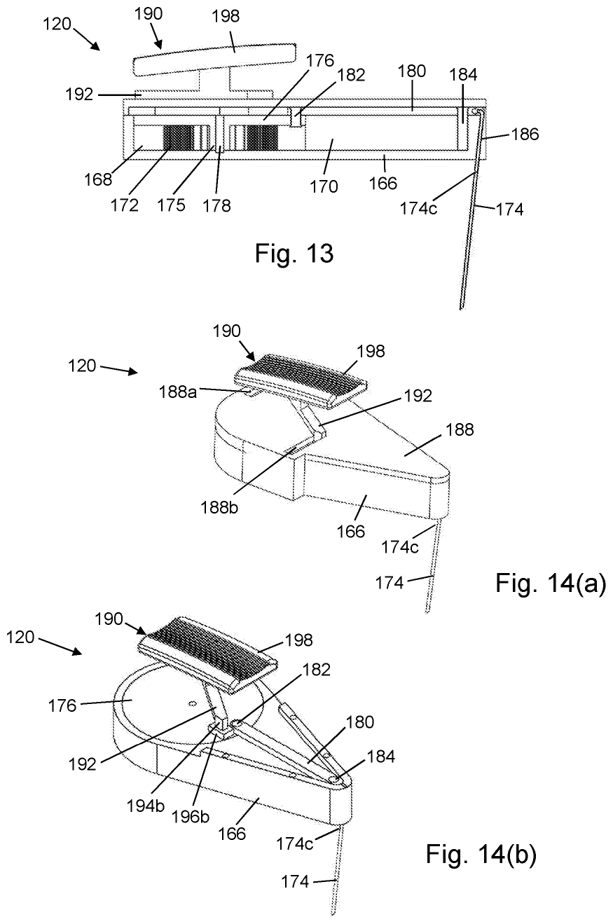

When the trigger component 190 is moved laterally to the second position, the legs 194a, 194b each slide laterally in their respective slots 188a, 188b. Now, the first blocking formation 196a moves clear of the first end of the shaft 180, allowing the disc 176 to rotate clockwise. This drives linear movement of the second end of the shaft 180, guided by the guide pin 184 and the guide slot 170, which feeds the cannula 174 through the channel 186 to extend from the bottom surface of the guide body 166. FIGS. 13 and 14 show the cannula insertion mechanism 120 after the cannula 174 has been deployed.

As can be seen most clearly in FIG. 14(b), when the trigger component 190 is in the second position, the second blocking formation 196b now engages with the first end of the shaft 180 to limit the extent of clockwise rotation of the disc 176 to approximately 180 degrees. This locks the cannula 174 in the extended position.

The cannula 174 is preferably of a superelastic material, such as nitinol. The cannula 174 may have a straight configuration when unconstrained, and bending of the cannula 174 in its initial stowed configuration and during deployment of the cannula 174 is accommodated by elastic deformation of the material so that the exposed part of the cannula 174 is straight after deployment.

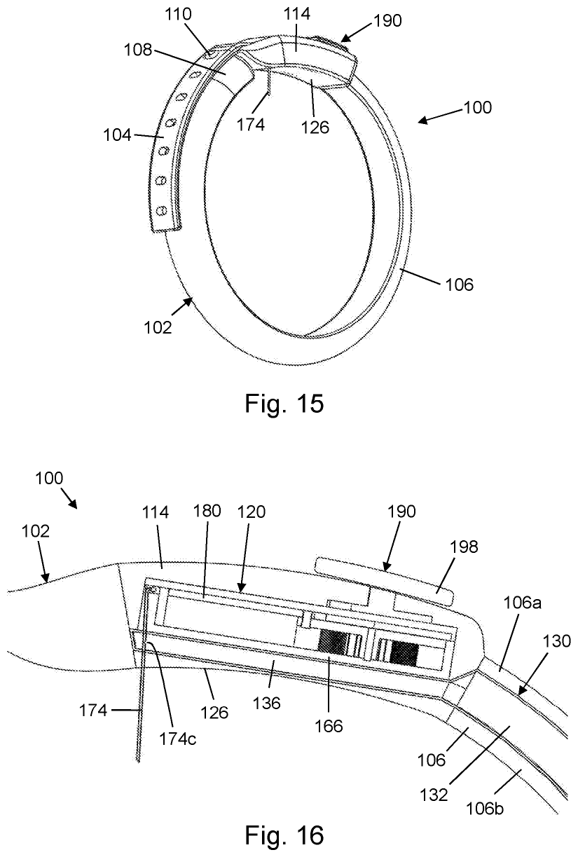

Operation of the cannula insertion mechanism 120 when the device 100 is fastened in place, with the contact surface 126 against the injection site, causes the cannula 174 to extend from the contact surface 126 as shown in FIGS. 15 and 16. In this way, the cannula 174 is inserted into the injection site. Preferably, the insertion depth of the cannula 174 is approximately 10 mm. The inclined angle of the channel 186 causes the cannula 174 to be deployed approximately perpendicularly to the injection site, taking into account the generally curved shape of the contact surface 126.

As can be seen most clearly in FIG. 16, upon deployment, the cannula 170 pierces the reservoir bag 130 to extend through the end chamber 136 of the bag 130. The cannula 174 includes a side orifice 174c positioned so that, after deployment of the cannula 174, the orifice 174c is disposed within the end chamber 136 of the bag 130 to open a pathway for the flow of medicament from the interior of the bag 130 to the injection site through the cannula 174. The end of the cannula 174 that is attached to the shaft 180 is closed to prevent misdirection of the medicament.

Because the medicament in the reservoir bag 130 is pressurised by the stretched band 106 of the holder 102, medicament is driven through the cannula 174 into the injection site. Said another way, medicament delivery is driven by the elastic potential energy stored in the stretchable elements of the holder 102. Medicament delivery continues as the elastic strain in the band 106 relaxes until the holder 102 has returned substantially to its initial shape to flatten the main part 132 of the bag 130 between the outer and inner walls 106a, 106b of the band 106.

As a result of the varying thickness of the outer wall 106a of the band 106 of the holder 106, the compressive force applied to the reservoir bag 130 is greatest closest to the fastener end 108 of the holder 102 and decreases with distance moving towards the housing 114. Consequently, the medicament is effectively squeezed from the filling end termination 134 of the bag 130, with the filling end 134 of the bag 130 emptying first. This helps to ensure that the whole dose of medicament is delivered through the cannula 174, particularly when the viscosity of the medicament is relatively high. In a variant of the device (not illustrated), the outer and inner walls 106a, 106b of the band 106 of the holder 102 each have constant thicknesses.

It will be appreciated that some medicament may remain in the bag 130 after delivery. In particular, a small quantity of medicament is likely to remain in the enlarged filling end termination 134 of the bag 130, and in the enlarged end chamber 136 of the bag 130 disposed below the cannula insertion mechanism 120. However, the volume of medicament that remains in the device 100 is predictable and repeatable between devices, and can therefore be accounted for when determining the dose volume delivered by the device 100.

Once delivery of the medicament is complete, the cannula 174 can be withdrawn from the injection site by sliding the trigger component 190 laterally from its second position (shown in FIG. 14(b)) back to its initial position (FIG. 12(b)). In doing so, the second blocking formation 196b is moved clear of the first end of the shaft 180, allowing the disc 176 to rotate clockwise, driven by the power spring 172, through another 180 degrees to cause the shaft 180 to retract the cannula 174 back into the housing 114. Further clockwise rotation of the disc 176 is then blocked by the first blocking formation 196a. In this state, the cannula 174 is once again stowed within the guide body 166 of the cannula insertion mechanism 120.

After withdrawal of the cannula 174, the device 100 can be removed from the patient by releasing the strap 104 from the catch 110. The device 100 can then be safely disposed of.

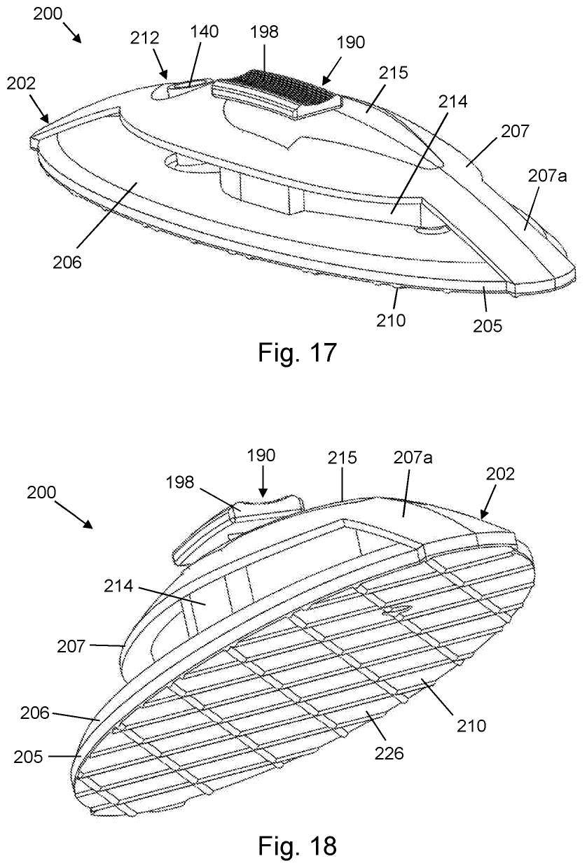

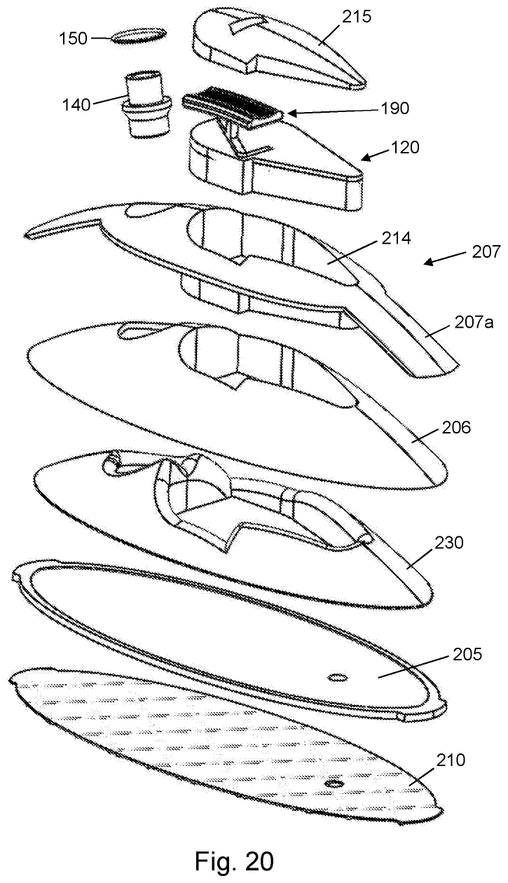

A device 200 according to a second embodiment of the invention is shown in FIGS. 17 to 22. Referring first to FIGS. 17 to 19, which show the device 200 in its unfilled state, and to FIG. 20, which is an exploded view of the device 200 after filling with medicament, the device 200 includes a body or holder 202 comprising a chassis tray 205 and a frame 207. A bottom surface of the chassis tray 205 carries an adhesive layer 210 and provides a contact surface 226 to be placed against the injection site in use. A removable backing layer (not shown) is applied to the adhesive layer 210 to protect the adhesive until the device 200 is ready for use.

The frame 207 includes leg portions 207a that attach to opposite sides of the chassis tray 205. The leg portions 207a support a housing 214 that is suspended above a central part of the chassis tray 207. A cannula insertion mechanism 120 of the type described above with reference to FIGS. 10 to 14 is disposed in the housing 214 and retained by a cover 215, with the button 198 of the trigger component 190 disposed above the cover 215.

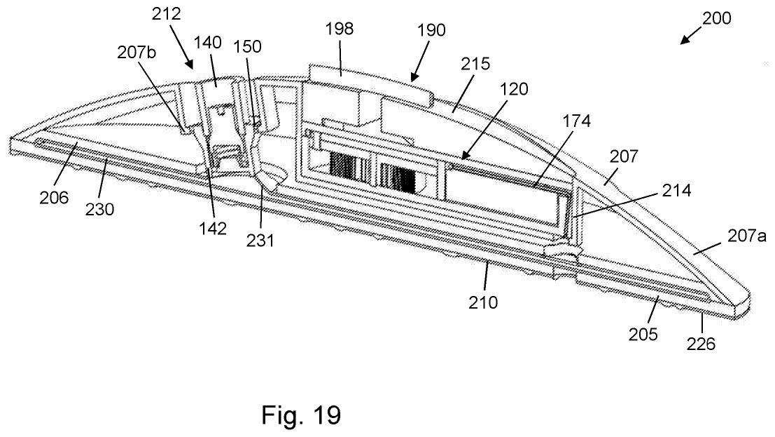

A stretchable elastic sheet 206 extends across the top of the chassis tray 205 and underneath the frame 207. The elastic sheet 206, which is preferably of a thermoplastic elastomer material, is affixed to the tray 205 around its periphery, for example by an adhesive. A reservoir bag 230, visible in FIGS. 19 and 20, is disposed between the upper surface of the tray 205 and the lower surface of the elastic sheet 206, so that a wall of the reservoir formed by the bag 230 is adjacent to the elastic sheet 206.

A valve 140, substantially as described above with reference to FIG. 7, is mounted in a recessed part 207b of the frame 207 and sealed with an O-ring 150 to provide a filling port 212 of the device 200. The valve 140 is mounted so that the outer end of the valve 140 is substantially flush with the upper surface of the frame 207.

As shown most clearly in FIG. 19, in its initial, unfilled state, the reservoir bag 230 is substantially flat, with the exception of a filling part 231 that extends upwardly from the top side of the bag 230 to form a seal around the inner housing part 142 of the valve 140.

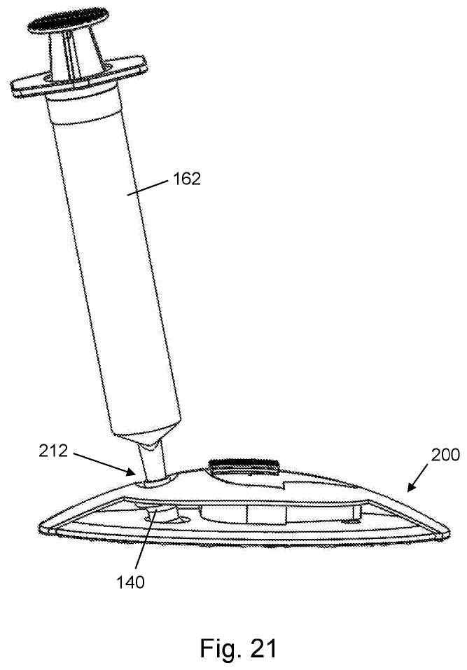

The reservoir bag 230 can be filled using a filling syringe 162 to transfer medicament into the bag 230 through the filling port 212, as shown in FIG. 21. After filling, the valve 140 closes to re-seal the interior of the bag 230.

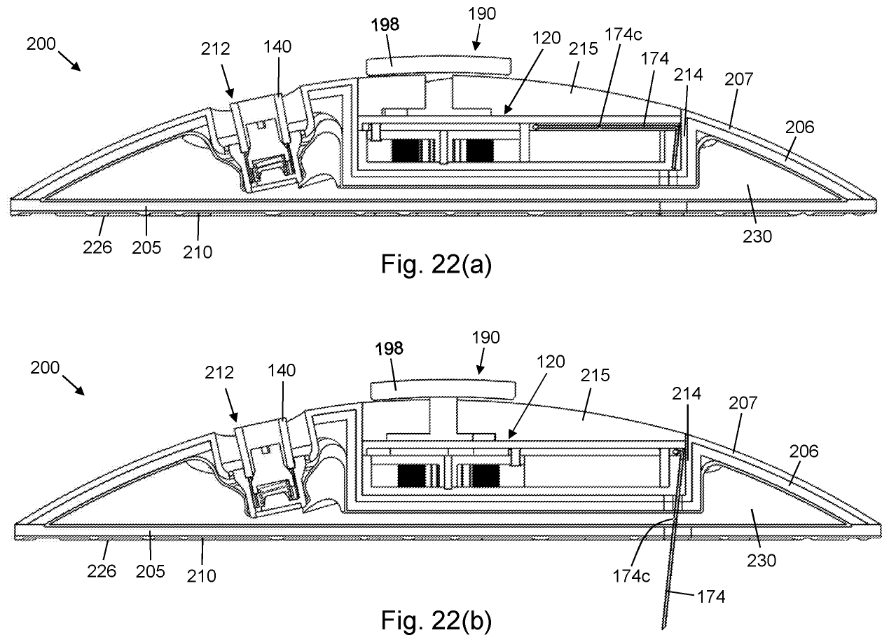

Transfer of medicament to the reservoir bag 230 causes the bag 230 to expand from its initial, flat state as shown in FIG. 19, to an expanded state as shown in FIGS. 20 and 22(a). Expansion of the bag 230 causes the elastic sheet 206 to stretch elastically into a shape that substantially conforms to the shape of the underside of the frame 207. The stretched material of the elastic sheet 206 applies a compressive force to the reservoir bag 230 to pressurise the medicament within the bag 230.

The device 200 can be attached to the injection site by removing the backing paper and applying the adhesive layer 210 of the contact surface 226 to the patient's skin. The medicament can be transferred to the device 200 either before or after the device 200 is attached to the injection site.

Once secured to the injection site, delivery of the medicament to the patient is triggered by operation of the cannula insertion mechanism 120 by moving the trigger component 190 from a first position to a second position, as described above.

As shown in FIG. 22(b), upon operation of the insertion mechanism 120, the cannula 174 extends to pass through apertures in the frame 207, the elastic layer 206, the chassis tray 205 and the adhesive layer 210 to protrude from the contact surface 226 and enter the injection site. In doing so, the cannula 174 pierces the reservoir bag 230, and the orifice 174c in the cannula 174 is moved into position in the interior of the bag 230. In this way, a flow path for medicament is established between the bag 230 and the injection site through the cannula 174.

Medicament is driven through the cannula 174 by the compressive force applied to the reservoir bag 230 by the stretched elastic sheet 206. Delivery of medicament continues until the elastic sheet 206 has relaxed back to its initial, flat configuration.

Once medicament delivery is complete, the cannula 174 can be withdrawn by moving the trigger component 190 back to the first position, and then the device 200 can be removed from the injection site and disposed of.

Devices according to the invention can be of any convenient size for the delivery of a desired volume of medicament. Because the devices can be left attached to the injection site for an extended period of time without user intervention, they are particularly suitable for the delivery of relatively large volumes of medicament (for example from approximately 2 mL to approximately 50 mL) over a relatively long time period (for example from approximately 10 seconds to approximately 1 hour), although smaller or larger volumes and shorter or longer delivery times are possible.

The properties of the elastic element of the device (i.e. the band 106 of the holder of the device of FIGS. 1 to 16, or the elastic sheet 206 of the device of FIGS. 17 to 22) can be selected as necessary to achieve a suitable delivery rate. In particular, the thickness and shape of the elastic element, the elasticity of the material and the mechanical constraints on the element can be selected as required. Other elastic element arrangements are possible. For example, the reservoir may be accommodated between two stretchable elastic sheets, or in a stretchable elastic balloon.

In other embodiments, the elastic element may be compressible. For example, in one arrangement, the holder includes a chamber for receiving the reservoir, and one or more compressible elements mounted in the chamber or forming part of one or more walls of the chamber. When the reservoir is filled with medicament, the reservoir presses against the compressible elements to hold the elements in compressive strain, and delivery of the medicament is driven by relaxation of the compression.

The material of the reservoir itself may be stretchable to accommodate the volume of medicament after filling, in which case the reservoir may act as an additional pressurising means for the medicament, or even as the only stretchable element. Alternatively, the reservoir may be of a flexible material that does not appreciably stretch when filled with medicament. In this case, the reservoir bag may be folded, pleated, rolled or otherwise collapsed when in its initial empty state, so that the bag can conform to accommodate the volume of medicament during filling.

The reservoir need not be in the form of a flexible bag. For example, the reservoir could instead be of a substantially rigid material, in which case the reservoir could comprise two or more parts or walls that are moveable with respect to one another to allow the reservoir to expand upon filling with medicament and then to contract during delivery under the force applied by the elastic element. In such cases, the reservoir may comprise one or more moveable pistons or one or more telescoping parts.

As described above, in some cases it may be desirable for the elastic strain energy stored in the elastic element to vary with position, so that the force applied to the reservoir is non-uniform. This allows the emptying behaviour of the reservoir to be controlled, for example to force the parts of the reservoir furthest from the cannula to empty first to avoid medicament being trapped in the extremities of the reservoir. In the embodiment illustrated in FIGS. 1 to 16, this behaviour is achieved by an elastic element with a thickness (or cross-sectional area) that increases with distance from the cannula. For a given strain, the elastic energy stored in a thicker part of the elastic element, and therefore the force applied to the reservoir, is higher. The same effect could instead be achieved by arranging the reservoir and the elastic element so that the strain induced in the elastic element when the reservoir is filled varies with position. For instance, in one variant, the elastic element is arranged so that it is spaced from the reservoir before filling by a variable gap. Remote from the cannula, the gap is small or zero, and the gap increases moving towards the cannula. In this way, when the reservoir is filled, the elastic element is subject to increasing deformation moving away from the cannula. Another possibility is to vary the elastic modulus of the elastic element with position.

The device may be supplied as part of a kit that also includes a pre-filled filling syringe containing the medicament to be delivered. Preferably, the reservoir is sized to accommodate all of the medicament in the filling syringe, so that the user can transfer the entire contents of the syringe to the device before use. It is however possible that the device could be filled from a syringe with a capacity that exceeds the capacity of the reservoir.

The filling syringe may be engageable with the valve using any suitable arrangement. For example, the slip-tip (Luer slip) arrangement of the illustrated embodiments could be replaced with a Luer lock arrangement or other suitable connector system. The valve itself may be of a suitable known type. The use of a needle-free filling syringe is preferable, since this reduces the risk of the medicament being delivered by direct injection from the filling syringe. However, it is conceivable that the filling valve of the device could include a pierceable septum, in which case the filling syringe may include a needle for piercing the septum to transfer the medicament to the device.

Whilst the illustrated devices are intended to be filled with medicament shortly before use, in other embodiments the reservoir is pre-filled with medicament during manufacture of the device. In these cases, the filling valve need not be accessible to the user after manufacture of the device, or could be omitted entirely.

Other arrangements for holding the device in position against an injection site are possible. For example, in devices with an arm band configuration as shown in FIGS. 1 to 16, the strap and catch arrangement could be replaced with a hook-and-loop fastening, an adhesive fastening, a strap and buckle arrangement, a clasp arrangement, a press-stud fastening or any other suitable fastening. A releasable fastening may be omitted entirely, and the device may be provided in the form of a ring that is stretchable or includes one or more stretchable parts to allow the device to be stretched over a limb and then remain in place. Although such devices have been described as arm bands, it will be understood that devices could be sized appropriately for use on a different body part, such as the leg, finger, neck or torso of a patient. Devices with a patch configuration, as shown in FIGS. 17 to 22, may also be fitted with a strap to assist in securing the device in position.

Any suitable cannula insertion mechanism may be provided. For example, insertion and/or retraction of the cannula may be driven by one or more tension or compression springs. Retraction of the cannula may be triggered automatically after the medicament dose has been delivered, for example by use of a timer device or a mechanical trigger that responds to emptying of the reservoir. It is also conceivable that insertion and/or retraction of the cannula could be performed manually by the user. Where the cannula is guided to bend by a channel, the channel can be arranged at any suitable angle so that the cannula extends at a suitable orientation from the contact surface upon deployment. In the illustrated examples, the channel causes the cannula to bend through an acute angle, but in other arrangements the cannula may bend through a right angle or through an obtuse angle. It is also possible that the cannula could remain straight in both the stowed and deployed positions. The cannula insertion mechanism described with reference to FIGS. 10 to 14 could conceivably be used in medicament delivery devices of types other than those described herein.

Further modifications and variations not explicitly described above are also possible without departing from the scope of the invention as defined in the appended claims.

1. A medicament delivery device for delivering a dose of medicament into an injection site, the device including:
an expandable reservoir for receiving the medicament;
a holder for receiving the reservoir;
an attachment for securing the holder to the injection site;
a cannula moveable from a stowed position in which the cannula does not project from the device to a deployed position in which the cannula provides fluid connection between the reservoir and the injection site; and
an elastic element;
wherein, in use, the elastic element is held under strain by the medicament in the reservoir, and delivery of the medicament through the cannula is driven by relaxation of the elastic element when the cannula is in the deployed position.

2. A device according to claim 1, wherein the reservoir comprises a flexible bag.

3. A device according to claim 1, wherein the elastic element is of an elastomeric material.

4. A device according to claim 1, wherein the reservoir comprises a wall at least partially defining an interior of the reservoir for receiving the medicament, and wherein the elastic element is disposed adjacent to the wall.

5. A device according to claim 1, wherein the elastic element is stretchable, such that the elastic element is held under tensile strain by the medicament in the reservoir.

6. A device according to claim 1, wherein the holder comprises the elastic element.

7. A device according to claim 1, wherein the cannula comprises a flexible needle.

8. (canceled)

9. A device according to claim 1, wherein the cannula comprises an orifice positioned such that the orifice is disposed within the reservoir when the cannula is in the deployed position.

10. A device according to claim 1, wherein the cannula is arranged to pierce the reservoir upon movement of the cannula from the stowed position to the deployed position.

11. A device according to claim 1, comprising a cannula insertion mechanism operable to move the cannula from the stowed position to the deployed position.

12-13. (canceled)

14. A device according to claim 11, wherein the cannula insertion mechanism comprises a power spring and a crank arrangement driven by the power spring and coupled to the cannula for moving the cannula between the stowed and deployed positions.

15. A device according to claim 14, comprising a trigger component having a first position in which the trigger component blocks movement of the crank arrangement with the cannula in the stowed position, and a second position in which the trigger component blocks movement of the crank arrangement with the cannula in the deployed position.

16. A device according to claim 1, wherein the attachment comprises an adhesive layer for securing the device to the injection site.

17. A device according to claim 1, wherein the attachment comprises an adjustable strap.

18. A device according to claim 1, wherein the elastic element comprises an elastic sheet.

19. A device according to claim 18, wherein the holder comprises a chassis, and wherein at least a part of the reservoir extends between the chassis and the elastic sheet.

20. A device according to claim 1, wherein the device comprises a wearable patch.

21. A device according to claim 1, wherein the device is elongate and flexible to be worn around a limb.

22. A device according to claim 21, wherein the elastic element comprises a tubular band, and wherein at least a part of the reservoir extends within the band.

23. A device according to claim 1, wherein the elastic element has a variable thickness to apply a driving force to the reservoir that varies with distance from the cannula.

24-27. (canceled)

28. A medicament delivery device for delivering a dose of medicament into an injection site, the device comprising a wearable patch including:
an expandable reservoir for receiving the medicament;
a holder for receiving the reservoir;
an attachment for securing the holder to the injection site;
a cannula moveable from a stowed position in which the cannula does not project from the device to a deployed position in which the cannula pierces the expandable reservoir to provide fluid connection between the reservoir and the injection site; and
a stretchable elastic sheet;
wherein at least a part of the expandable reservoir is disposed between the elastic sheet and a chassis of the holder;
and wherein, in use, the elastic sheet is held under strain by the medicament in the reservoir, and delivery of the medicament through the cannula is driven by relaxation of the elastic sheet when the cannula is in the deployed position.

29. A medicament delivery device for delivering a dose of medicament into an injection site, the device being elongate and flexible to be worn around a limb and including:
an expandable reservoir for receiving the medicament;
a tubular holder for receiving at least a part of the reservoir, the holder comprising a stretchable elastomeric material;
an attachment for securing the holder to the injection site; and
a cannula moveable from a stowed position in which the cannula does not project from the device to a deployed position in which the cannula provides fluid connection between the reservoir and the injection site;
wherein, in use, the tubular holder is held under strain by the medicament in the reservoir, and delivery of the medicament through the cannula is driven by relaxation of the tubular holder when the cannula is in the deployed position.
